(19) United States
(12) Patent Application Publication
(10) Pub. No.: US 2005/0234509 A1
(43) Pub. Date: Oct. 20, 2005

(54) Center joints for PFO occluders

(75) Inventors: David R. Widomski, Wakefield, MA (US); Carol A. Devellian, Topsfield, MA (US)

Correspondence Address: WILMER CUTLER PICKERING HALE AND DORR LLP, 60 STATE STREET, BOSTON, MA, 02109, US

(73) Assignee: MMT Medical, Inc., Boston, MA (US)

(21) Appl. No.: 11/093,360

(22) Filed: Mar. 30, 2005

Related U.S. Application Data
(60) Provisional application No. 60/557,486, filed on Mar. 30, 2004.

(51) Int. Cl. A61B 17/08

(52) U.S. Cl. 606/213

(57) ABSTRACT

The disclosed invention provides several alternative embodiments of devices that include center joints. The devices are adapted for use in a Patent Foramen Ovale (PFO) closure device. In one embodiment of the invention the center joint may be flexible and may have locking capacity to allow a PFO closure device to be delivered in a reduced profile and then locked into a deployed configuration. When the center joint is flexible the device can conform to the anatomy of the PFO. The device may include ends that cooperate with the center joint to allow a clamping function on the tissue to close the PFO. In another embodiment, the center joint expands to fill some or all of the PFO tunnel. In still another embodiment the center joint is designed to stretch the tunnel from side to side so that the inner surface of the tunnel collapses onto itself. In still another embodiment, the center joint can be inflatable, e.g., as with a balloon, so that the PFO tunnel is filled when the balloon is inflated.

### CROSS REFERENCE

This application claims the benefit of provisional application U.S. Ser. No. 60/557,486 filed on Mar. 30, 2004.

### BACKGROUND

The present invention relates generally to an occlusion device for the closure of physical anomalies like septal apertures, such as patent foramen ovale and other septal and vascular defects.

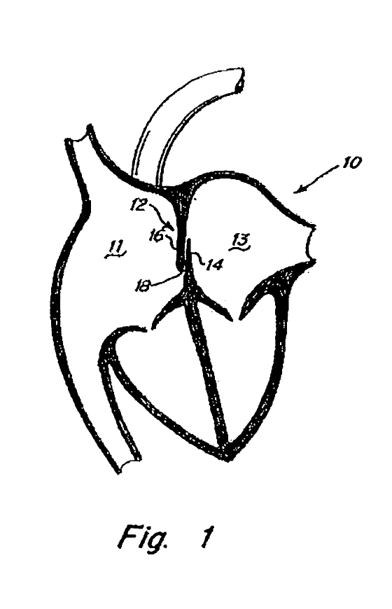

A patent foramen ovale (PFO), illustrated in FIG. 1, is a persistent, one-way, usually flap-like opening in the wall between the right atrium 11 and left atrium 13 of the heart 10. The PFO is typically an oblique opening between the tissue that separates the left atrium from the right atrium and is sometimes called a “tunnel”. Because left atrial (LA) pressure is normally higher than right atrial (RA) pressure, the flap usually stays closed. Under certain conditions, however, right atrial pressure can exceed left atrial pressure, creating the possibility that blood could pass from the right atrium 11 to the left atrium 13 and blood clots could enter the systemic circulation. It is desirable that this circumstance be eliminated.

The foramen ovale serves a desired purpose when a fetus is gestating. Because blood is oxygenated through the umbilical cord, and not through the developing lungs, the circulatory system of a heart in a fetus allows the blood to flow through the foramen ovale as a physiologic conduit for right-to-left shunting. After birth, with the establishment of pulmonary circulation, the increased left atrial blood flow and pressure results in functional closure of the foramen ovale. This functional closure is subsequently followed by anatomical closure of the two over-lapping layers of tissue: septum primum 14 and septum secundum 16. However, a PFO has been shown to persist in a number of adults.

The presence of a PFO is generally considered to have no therapeutic consequence in otherwise healthy adults. Paradoxical embolism via a PFO is considered in the diagnosis for patients who have suffered a stroke or transient ischemic attack (TIA) in the presence of a PFO and without another cause of ischemic stroke. While there is currently no definitive proof for a cause-effect relationship, many studies have confirmed a strong association between the presence of a PFO and the risk for paradoxical embolism or stroke. In addition, there is significant evidence that patients with PFO who have had a cerebral vascular event are at increased risk for future, recurrent cerebrovascular events.

Accordingly, patients with an increased future risk are considered for prophylactic medical therapy to reduce the risk of a recurrent embolic event. These patients are commonly treated with oral anticoagulants, which have the potential for adverse side effects, such as hemorrhaging, hematoma, and interactions with a variety of other drugs. The use of these drugs can alter a person's recovery and necessitate adjustments in a person's daily living pattern.

In certain cases, such as when anticoagulation is contraindicated, surgery may be necessary or desirable to close the PFO. The surgery would typically include suturing a PFO closed by attaching septum secundum to septum primum. This sutured attachment can be accomplished with either an interrupted or a continuous stitch and is a common way a surgeon shuts a PFO under direct visualization.

Umbrella devices and a variety of other similar mechanical closure designs, developed initially for percutaneous closure of atrial septal defects (ASDs), have been used in some instances to close PFOs. These devices have the potential to allow patients to avoid the potential side effects often associated with anticoagulation therapies and the risks of invasive surgery. ASD devices are designed to occlude a hole and as a result many lack anatomic conformability to the PFO flap-like anatomy. That is, when inserting an ASD device to close a PFO, the narrow opening and the thin flap may impede proper deployment. Even if an occlusive seal is formed, the device may be deployed in the heart on an angle, which could leave some components not securely seated against the septum, thereby risking thrombus formation due to hemodynamic disturbances

PFO closure devices typically consist of three basic components: elements on each side of the PFO to keep the PFO closed and a center joint to hold the elements together. The elements at each side of the PFO can have a variety of configurations such as clips, petals umbrella, discs or spiral, which close the PFO to prevent blood from passing through the PFO and allow a therapeudic response to allow tissue to grow and close the PFO. The center joint connects the two ends and is designed to pass through the PFO. The characteristics of these three components must allow these devices to be delivered in a reduced profile, retrieved if necessary, and, once deployed at the delivery site, keep the PFO closed.

### SUMMARY OF THE INVENTION

The present invention provides several alternative embodiments of center joints that are adapted for use in a PFO closure device. In one embodiment of the invention the center joint may be flexible and may have locking capacity to allow a PFO closure device to be delivered in a reduced profile and then locked into a deployed configuration. When the center joint is flexible the device can better conform to the anatomy of the PFO. The device may include ends that cooperate with the center joint to allow a clamping function on the tissue to close the PFO. In another embodiment, the center joint expands to fill some or all of the PFO tunnel. In still another embodiment the center joint is designed to stretch the tunnel from side to side so that the inner surface of the tunnel collapses onto itself. In still another embodiment, the center joint can be inflatable, e.g., as with a balloon, so that the PFO tunnel is filled when the balloon is inflated.

In other embodiments, the center joints are designed to modify the surrounding geometry of the PFO. For example, the center joint may be designed to open up the tunnel so that alternative treatments would be possible.

In some configurations, the center joint may be used with a variety of configurations at each end. For example, a center joint according to the invention may be used with various disc or umbrella shaped occlusion devices. The occlusion devices may have spirals, star-shaped, clipped or umbrella-shaped ends that are adapted to close the PFO and secure the device in place. Alternatively, some embodiments of the center joint could be used without any occlusion “ends” (such as umbrellas or star-shaped ends) and to close the PFO tunnel by modifying the shape of the tunnel from within the tunnel.

These and other features of the invention will be understood with reference to the Figures and the accompanying detailed description of the invention.

FIG. 1 is an illustration of a human heart showing the location of a PFO.

FIGS. 2a is an illustration of an embodiment of a PFO closure device with center joint with flexible suture locking mechanisms.

FIG. 2b is an illustration of an embodiment of a PFO closure device with a center joint and illustrating distal and proximal ends of the device.

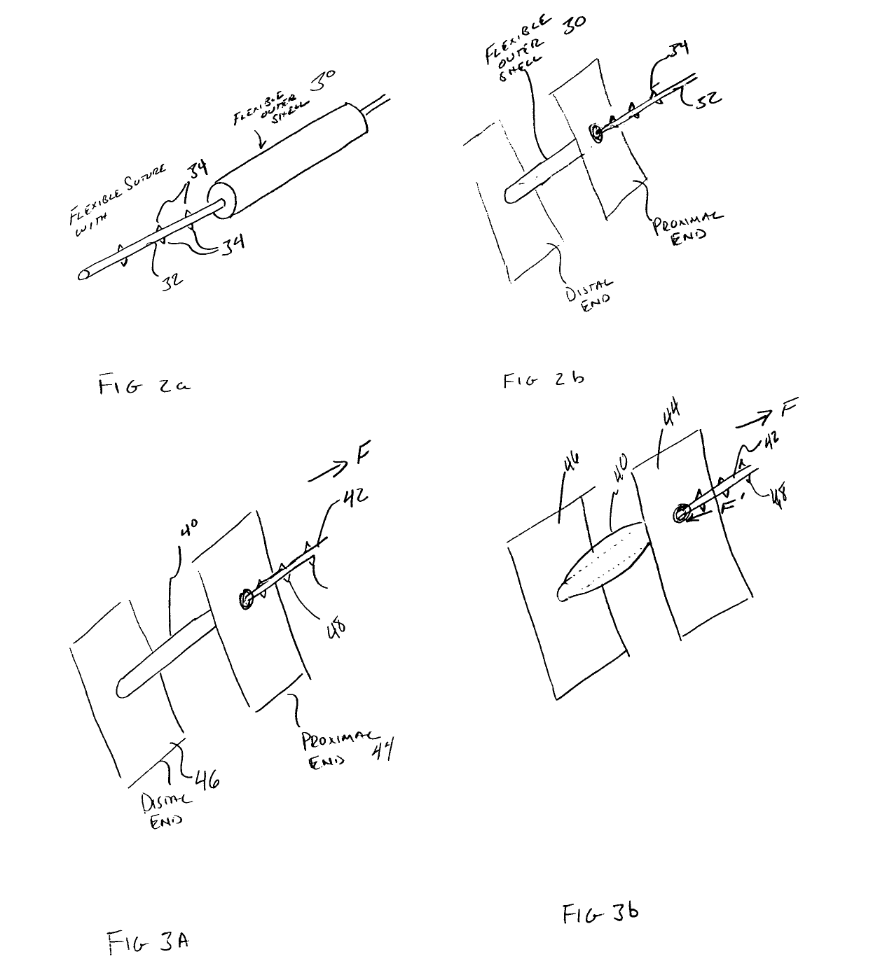

FIGS. 3a and 3b illustrate embodiments of a PFO closure device according to other aspects of the invention.

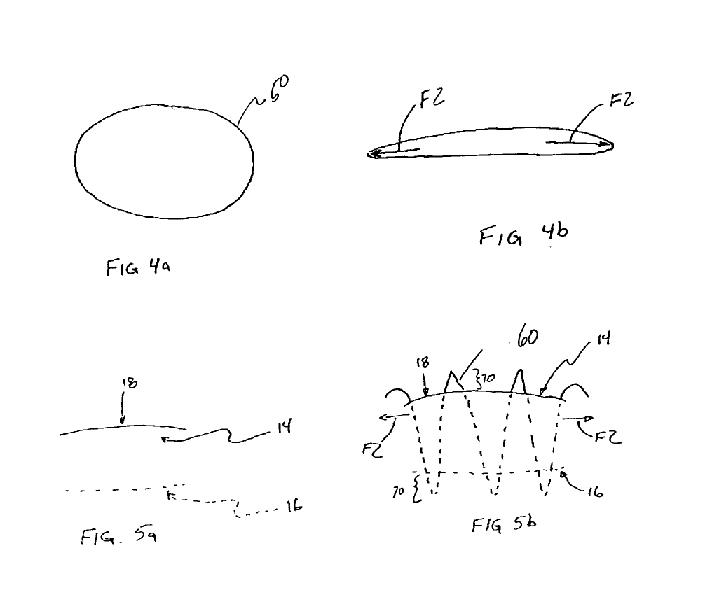

FIGS. 4a and 4b schematically illustrate the effect of forces on the PFO that would tend to close a PFO by stretching in the transverse direction.

FIGS. 5a and 5b illustrate alternative structures that can be used as or with a center joint to modify the configuration of the PFO tunnel when the device is deployed in the body.

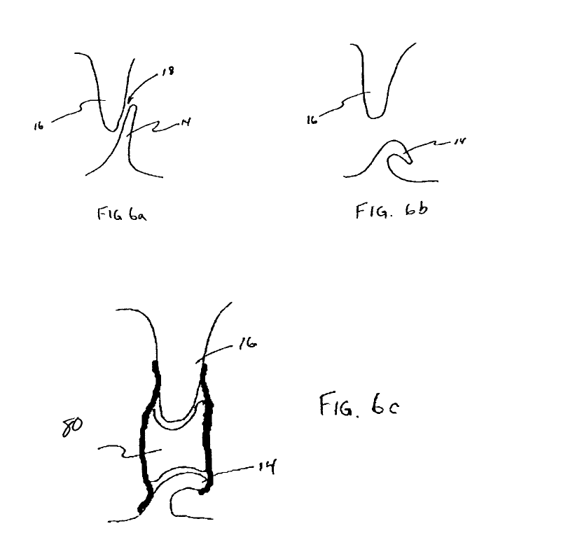

FIGS. 6a-6c illustrate another concept for a center joint which can be used to close a PFO.

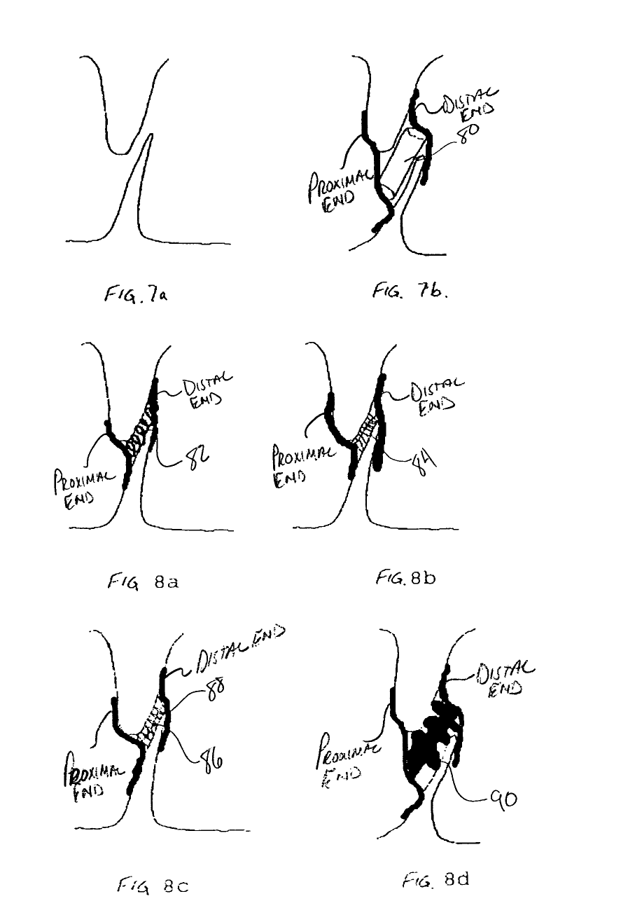

FIGS. 7a and 7b illustrate still another embodiment of a device according to the invention for closing a PFO.

FIGS. 8a-8d illustrate still another embodiment of the device according to the invention that promotes irritation between the septal tissue.

### DESCRIPTION OF INVENTION

The present invention provides a device for occluding an aperture within body tissue. In particular and as described in detail below, the occluder of the present invention may be used for closing a PFO in the atrial septum of a heart. Although the embodiments of the invention are described with reference to a PFO, one skilled in the art will recognize that the device and method of the present invention may be used to treat other anatomical conditions. As such, the invention should not be considered limited to any particular anatomical condition.

FIG. 1 illustrates a human heart 10, having a right atrium 11 and a left atrium 13. The atrial septum 12 includes septum primum 14, septum secundum 16, and a passage 18 between the right 11 and left 13 atria. The anatomy of the septum varies widely within the population. In many people, septum primum 14 extends to and overlaps with septum secundum 16. The septum primum 14 may be quite thin. When a PFO is present, there is a chance that blood could travel through the passage 18 between septum primum 14 and septum secundum 16 (referred to as “the PFO tunnel”). The flow of blood through the passage can lead to adverse health consequences.

Devices that are used to treat a PFO may be adapted to be delivered through a catheter and deployed at the PFO using a percutaneous approach. As such, the devices typically have a reduced profile configuration in the catheter and a deployed configuration at the delivery site. A device that is deployed typically has three basic components: the ends, which are disposed on each side of the PFO and a connector, sometimes called a center joint, that connects the ends. The center joint is typically disposed in the PFO tunnel.

This application describes various center joints that are used, alone or in combination, in devices for repairing PFOs and similar anatomical configurations. The center joints could be used with ends that extend beyond the PFO tunnel or, alternatively, can be used singly and remain largely within the PFO tunnel. Overall, a desirable configuration should close a PFO and remain secure at the delivery location after deployment.

The complex geometry of a PFO leads to many potential variations of such center joints. This disclosure describes six types of center joints as follows:
Flexible center joint with clamping or locking capabilities
Flexible center joint that expands
Center joint designed to selectively stretch the tunnel
Center joint designed to disrupt the surrounding geometry
Center joint designed to open up the tunnel
Center joint designed to irritate the surrounding tissue

The sections below describe each of these categories in more detail. The concepts set forth in these categories focus mainly on the coupling between the proximal and distal ends of an occluder. Although the descriptions necessarily refer to the occluders to which the couplings are attached, the descriptions do not address details of specific occluders. Instead, the descriptions address the coupling of a general occluder apparatus.

Flexible center joint with clamping or locking capabilities—A flexible center joint can extend through the PFO tunnel and connect two ends (occluder surfaces) of an occluding member. The two ends can provide some biased force against the PFO tissue to close a PFO defect. The device may be permanent or temporary. The clamping down provides additional holding force to close the defect. A flexible center joint aids in the closure of complex tunnels by allowing the occluder surfaces to deploy at non-parallel orientations. Specifically, a flexible center joint can allow for a more complete coverage of the occluder over the PFO tissue. The flexible center joint may be rigid enough to allow for force-transmission during delivery. In other embodiments the occluder itself may provide the column strength for the system to be delivered with the flexible center joint not transmitting the force to the distal side of the occluder.

FIG. 2a shows a center joint including a flexible outer shell 30 with a flexible suture threaded through a center channel of the outer shell. The flexible suture 32 includes locking mechanisms 34, i.e., barbs or triangular teeth along its length that engage the end of the outer shell 30. These locking mechanisms allow the suture 32 to move relatively freely when pulled in one direction, and resist movement when pulled in the opposite direction. FIG. 2b shows the center joint of FIG. 2a combined with occluder components disposed at the distal and proximal ends of the center joint.

The flexible outer shell could be made from a variety of different biocompatible materials. One suitable material would be Poly Vinyl Alcohol (PVA) another suitable material may be polyurethane foam. The flexibility of the material should allow for tight bending radii so that the device conforms to the anatomy of the PFO in large part due to the flexibility of the center joint. The center joint should have enough stability so that it will stay in place within the PFO tunnel. The center joint is also able to apply a tensile stress to the ends so that the ends are pulled together. In one embodiment, the force is applied by a user. Also, the maximum extension (or compression) of the device could be limited so that the device is not loose (to too tight) on the septum.

The locking mechanism described above provides sufficient compressive force between the two ends. The combination of a flexible center joint and a locking mechanism allows the closure device to conform to the anatomical configuration of the PFO while allowing a compressive force to be imparted by the closure device.

The flexible outer shell of the center joint is illustrated as circular, it could, however, have any cross-sectional shape. In particular, a flat cross sectional shape may allow for a more complete closure of the PFO. Moreover, the flexible outer shell may be designed to conform to the anatomical path of the PFO tunnel. In such a configuration, the flexible outer shell will conform to the anatomy by flattening out, for example, at a narrow part of a PFO. Alternatively, a flexible spongy material may be used to allow the PFO tunnel to compress at any narrowing of the PFO.

The flexible center joint may also be a membrane which is constructed of flexible elastomeric material. The stability of the material can be modified by using strands of suture threads embedded within or attached to the membrane material. In that manner, the material may be more flexible in one dimension and more resistant to stretching in another.

Flexible Center Joint That Expands—A center joint 40 that expands, in addition to being flexible and providing a clamping force, is desirable in closing a PFO defect. A flexible center joint that can expand to fill, partially or fully, the middle region of a PFO will aid in closing the defect. The “occlusive” ends 44, 46 may be of any of a variety of suitable configurations. One material that is suitable for this embodiment is polyvinyl alcohol, although any biocompatible material that swells could be used. Additionally, the swelling could be caused by the absorption of a liquid (e.g., water or blood) or a chemical reaction.

One way to expand the center joint is to use an embedded suture 42 or wire in a flexible center joint. Pulling the suture (or wire) as illustrated by the arrow “F” from one end of the center joint while the suture (or wire) is anchored at the other end of the center joint provides a compressive force on the outer shell of the flexible center joint, as shown in FIG. 3a. Of course, there would need to be some force F′ established to allow for the axial length of the occluder be shortened. The force F′ may be applied by the distal end of a catheter (not shown). As this compressive force increases by continuing to pull on the suture (or wire), the center joint will bulge outward, away from the center axis of the outer shell, causing the center joint to expand, as shown in FIG. 3b. The dotted lines in FIG. 3b indicates the unexpanded center joint. The locking members 48 allow the occluder to be axially shortened incrementally. The locking members are sized lock in position at the proximal end of the occluder. The expansion of the centering joint can also assist in the centering of the device within the PFO. This can be facilitated by having different locations in the device have variable swelling properties, e.g., rate of swelling, extent of swelling.

Another way to expand the center joint is by dilating a balloon. Such a balloon may be disposed within the center joint, or the walls of the balloon can actually be the center joint. The balloon may be constructed from compliant or non-compliant material. One material particularly suited for use would be PEBAX® material. The center joint may be filled up with many different substances ranging from gas, liquid, polymer, epoxy, and biological material. The center joint may be designed to leach out the filling substance over time to the surrounding tissue.

The balloon can be designed with a variety of non-spherical shapes. In particular, a balloon shaped to correspond to the shape of a PFO tunnel provides a significant occlusion mechanism. The balloon would have a length that is sufficiently long enough to provide for a significant occlusion surface area for the PFO. Additionally, the shaped balloon, during inflation should only have slight expansion in the widest dimension of the balloon.

The outer surface of the balloon and/or center joint may include features that attach to the surrounding tissue, so that the balloon can expand, attach to the tissue, and then contract so as to pull the tissue together. The center joint may also expand in a similar fashion to a tampon or sponge device being inserted into the body.

This type of expanding center joint can be used alone or in conjunction with distal and/or proximal occluder components.

Center joint designed to selectively stretch a PFO defect—A center joint that changes the geometry of the tunnel to fit a specific, predetermined shape defined by the center joint is also useful for PFO closure. As an example, a center joint that stretches a tunnel with a round or slightly elliptic opening 50, as shown in FIG. 4a, by applying force F2 to opposite sides of the opening, as shown in FIG. 4b, would elongate the tunnel cross section to a narrow slit. A variety of structures could be used to apply the force such as wires or membranes that are used to stretch the tunnel along the width of the tunnel to urge the sides in closer contact.

Another example is a spring system 60 disposed within the PFO passage 18 that relaxes to expand at the sides of the PFO and force the septum primum 14 and the septum secondum 16 together, as shown in FIGS. 5a and 5b. In this particular example, the spring system 60 is shaped in a “zig-zag” pattern. The transitions can be rounded bends or relatively sharp bends, and the ends can be fabricated to attach to the ends of the defect.

The center joint may be composed of a shape memory metal, a shape memory polymer, and can incorporate a material that creates a biological response, for example a growth factor to encourage healing of the contacting tissues.

This type of device can be used without ends that provide compressive force to the PFO tunnel. As illustrated, the center joint can extend slightly beyond the PFO overlap as identified by reference numeral 70. Alternatively, the center joint may be entirely within the PFO. A variety of shapes may be suitable for such a device such as undulating curves.

Center joint designed to disrupt the surrounding geometry—A center joint that disrupts the geometry of the defect is useful for simplifying the complex geometry of the PFO tunnel/flap. As an example, a center joint designed to deform septum primum 14 as shown in FIG. 6A by pushing primum 14 out of the way as shown in FIG. 6b, creating a simple ASD (hole) out of a complex PFO. As a result, the closure device would be easier to produce and deploy, as shown in FIG. 6c. The arms that are useful to secure the device are illustrated with heavy (wider) lines.

Center joint designed to open up the tunnel—A PFO can be closed by increasing blood flow to the tunnel. This counter-intuitive method works for many reasons including: stimulation of growth factor, increase thrombosis build up, or producing a blood clot. FIGS. 7a and 7b illustrate this technique with a cylindrical center joint designed to open and expand the PFO tunnel. Although this example shows a cylindrical center joint 80, other shapes (e.g., planer, hexagonal cross section or other polygonal cross section center joints) are also suitable for this technique. The arms are illustrated with heavy (wide) lines.

Center joint designed to irritate surrounding tissue—A center joint that irritates the surrounding tissue is conducive to PFO closure. Irritating the tissue induces an inflammatory response or a biological response that will aide tissue in growth. The center joint can be shaped, or include features on its exterior, to irritate the adjacent tissue. Examples of shapes and/or features that provide such irritation are spiral coils, sharp-edged polygons and various sized bristles. Also, there may be other types of materials that could cause irritation. For example, there are chemicals that cause irritation, such as cod liver oil, that could be used.

These are illustrated FIGS. 8a-8d. As illustrated, various types of irritation devices are used with the center joint. For example, in FIG. 8a, a coil 82 is used to cause irritation. In this example, the outer surface of the coil rubs against the heart tissue and as a result of the rubbing, a tissue growth response occurs. The spring can also pulls the ends together to provide a more occlusive cover to the opening. FIG. 8b illustrates the use of polygons (which may or may not have a sharp edge) 84 that are attached to the center joint wire. FIG. 8b also illustrates a configuration where the occluder includes a wire in the center joint between the two ends of the occluder. FIG. 8c illustrates the use of a coil between the two ends of the occluder. The coil may have spring-like properties that can urge the ends toward one another when the device is deployed. Additionally a wire 88 is used to attach the ends so that if the device requires removal or redeployment the wire will allow the device to be pulled into a recovery catheter. The springs may be compressed and stacked during delivery which enhances delivery of the occluder through the catheter (e.g., pushability) and allows for an compact delivery. The center joint also includes a wire that limits the amount of distance that the coil can extend. Thus the configuration provides a means to retrieve occlusive member while allowing for a spring compression force between the occluder ends. FIG. 8d is an illustration of the device with bristles 90 that are used to cause irritation. Also, as apparent from FIG. 8d, various types of occluders can advantageously use irritants. For example, a device the separates the PFO can also irritate the inner surface of the PFO sufficiently to promote a tissue growth response.

1. An occlusive device comprising:
a first portion adapted to be on a first side of an anatomical defect, a second portion adapted to be on a second side of an anatomical defect, and
a center joint between the first and second portions that joins the first and second portions, wherein at least a portion of the center joint is adapted to pass through an anatomical defect and the center joint is adapted to expand radially when the axial dimension of the center joint is reduced.

2. The occlusive device as recited in claim 1, further comprising a locking mechanism that holds the occlusive device in an expanded configuration.

3. The occlusive device as recited in claim 2, wherein the locking mechanism further comprises a flexible strand with locking members and a passageway in the occlusive member through which the flexible strand is disposed, wherein the passageway is adapted to allow the strand to move freely through and lock the locking member by not allowing it to move freely though.

4. The occlusive device as recited in claim 2, wherein the strand includes multiple locking members that allows axial dimension of the center joint to be controlled during the deployment process.

5. The occlusive device as recited in claim 2, wherein the strand includes multiple locking members that allows the width of the center joint to be controlled during the deployment process.

6. The occlusive device as recited in claim 5, wherein the occlusive device is configured to be used as a PFO closure device for closing a PFO tunnel and the width of the center joint is controlled by the locking member so that the center joint expands into the PFO tunnel.

7. An occlusive device that is adapted to close a passageway that has a length and a width comprising:
a device adapted to fit within the passageway along at least a portion of the length, the device also having a configuration that imparts a force across the width of the passageway to close the passageway.

8. The occlusive device of claim 7, wherein the occlusive device includes a spring member that imparts a force across the passageway.

9. The occlusive device of claim 8 wherein the passageway is a PFO tunnel and the occlusive device is adapted to spread the tunnel along the width dimension so that the sides of the tunnel for therapeutic benefit.

10. The occlusive device of claim 9 further comprising a first end and a second end, the first end adapted to be disposed on one side of the passageway and the second end adapted to be disposed on the other side of the passageway.

11. The occlusive device of claim 10, wherein the device further comprises a zig-zag configuration to allow the device to expand the width of the passageway.

12. The occlusive device of claim 11 wherein the length of the zig-zag configuration extends substantially the entire length of the passageway.

13. A method of occluding a PFO tunnel that has a length and a width comprising the steps of:
inserting at least a portion of a device into the PFO tunnel, and
expanding the width of the tunnel with the device.

14. The method of occluding a PFO tunnel recited in claim 13, wherein the steps of expanding the width of the tunnel includes flattening the tunnel.

15. The method of occluding a PFO tunnel recited in claim 13, wherein the steps of expanding the width of the tunnel is performed with a zig-zag shaped occluding device.

16. The method of occluding a PFO tunnel recited in claim 15, wherein the inserting step is performed using a catheter.

17. An occlusive device comprising:
a first portion adapted to be on a first side of an anatomical defect, a second portion adapted to be on a second side of an anatomical defect, and
a center joint between the first and second portion that joins the first and second portions, wherein at least a portion of the center joint is adapted to promote a healing response in the anatomical defect.

18. The occlusive device recited in claim 17 further comprising a first side and a second side each adapted to be disposed on one side of an anatomical defect, wherein the center joint is in the form of a helical configuration and a wire strand between the first end and the second end to limit the distance between the two ends.

19. The occlusive device recited in claim 18, wherein the wire strand between the first end and the second end is with the spring.

20. The occlusive device recited in claim 19, wherein the spring provides force to pull the ends together once the device is deployed in the anatomical defect.

21. The occlusive device recited in claim 20, wherein the center joint includes both the spring and the wire strand and the center joint is adapted to promote a tissue healing response.
